(19) United States
(12) Patent Application Publication
(10) Pub. No.: US 2025/0208160 A1
(43) Pub. Date: Jun. 26, 2025

(54) AUTOMATED BUFFER EXCHANGE WITH GEL FILTRATION TIPS ON PRESSURE-FIT AUTOMATED LIQUID HANDLERS

(71) Applicant: Integrated Micro-Chromatography Systems, Inc., Irmo, SC (US)

(72) Inventors: Lim Andrew Lee, Columbia, SC (US); Patrick Alexander Kates, Lexington, SC (US); Brian Todd Mullis, Columbia, SC (US)

(21) Appl. No.: 18/392,791

(22) Filed: Dec. 21, 2023

(51) Int. Cl. G01N 35/10; C12N 15/10

(52) U.S. Cl. CPC G01N 35/1016; C12N 15/101; G01N 35/1002; G01N 2035/103; G01N 2035/1053

(57) ABSTRACT

The disclosure provides kits and methods for using buffer exchange pipette tips with pressure-fitted automated liquid handlers to exchange a sample of interest, such as a macromolecule, into a buffer of interest.

### BACKGROUND OF THE INVENTION

An analyte may require a buffer system conducive to formulation, assaying, or stability testing that is different from the buffer(s) used in its purification. Removal of salts and potential small-molecule contaminants is necessary for a bevy of biological assays associated with biotherapeutics and biomolecules. Adjustment of pH or replacement of the buffering salt may bestow additional advantages in stability. Failure to place a biomolecule in an appropriate buffer system may lead to chemical instabilities such as but not limited to deamidation, oxidation, or hydrolysis. There are several conventional approaches to buffer exchange including dialysis, centrifugal filtration, and column chromatography. While each method is effective, these methods are time consuming, require manual intervention or specialized equipment such as centrifuges or liquid chromatography systems, and are limited in their throughput capabilities.

Dialysis uses a semi-permeable membrane to facilitate selective diffusion of molecules in solution based on size. The pore size of the membrane determines the effective size, or molecular-weight cutoff (MWCO), of molecules that will be able to cross the membrane. Small molecules, such as most buffers or salts, can pass freely across the membrane and reach an equilibration concentration over the entire volume. Because this is equilibration driven, the dilution factor of the salt will be dependent on the total volume of the container in relation to the total volume inside the dialysis chamber. Large molecules above the MWCO are unable to diffuse across the membrane and thus remain inside of the dialysis chamber. As the volume inside of the chamber is restricted, the only dilution effect is related to the initial sample volume relative to the dialysis chamber volume. Dialysis chambers come in several forms, including membranes and cartridges (see e.g., U.S. Pat. Nos. 3,976,576 and 5,503,741). In general, traditional dialysis is effective, but requires large volumes of diluent relative to sample. Moreover, as volumes increase, time required to reach equilibration also increases.

Similar to dialysis, diafiltration separates molecules based on size. While dialysis utilizes passive diffusion throughout a solution, diafiltration uses an external pressure such as positive pressure or centrifugal forces, to push the material through a membrane. Several examples of such devices include centrifugal filters (see e.g., U.S. Pat. No. 8,357,296), continuous flow devices (see e.g., US Patent Publication No. 2017/0056825), tangential flow filtration processes (see e.g., U.S. Pat. No. 5,256,294), or combined automated diafiltration/diafiltration units (commercially available from e.g., Unchained Labs, Big Tuna). Because diafiltration uses positive pressure, the initial volume of sample can be reduced, resulting in a more concentrated sample.

Another common form of buffer exchange is to use column chromatography. Separation of analytes by either their molecular size or hydrodynamic volume using a column is referred to as size-exclusion chromatography (SEC). Removal of small moieties such as buffers and salts is commonly referred to as “desalting.” SEC is further differentiated into two procedures, gel permeation chromatography (GPC) and gel filtration chromatography (GFC). Both chromatographic techniques utilize inert resins comprised of a base material such as agarose, cellulose, dextran, polystyrene, polyacrylamide, polyacrylate, Sephadex, or Sepharose. Several commercial versions of this include: Sephadex, (cross-linked dextran epichlorohydrin), Bio-gel P (polyacrylamide), and Sepharose (agarose). Each inert resin is porous in nature. As a solution passes over the porous material, molecules smaller than the MWCO of the pores can become trapped within the pores, resulting in a longer traverse time for smaller molecules. The primary difference between GPC and GFC is that GPC utilizes organic solvents in the mobile phase. GFC uses aqueous solvents as a mobile phase and is primarily used in the buffer exchange of biological molecules.

Unlike dialysis where there is a strict MWCO to cross the membrane, SEC utilizes a continuous series of porous materials to provide a gradient of separation. As such, like in dialysis, it is possible to separate large molecules from small molecules, but unlike dialysis it is possible to fractionate between samples as well. The separation of large molecules from very small ones is tantamount to “buffer exchange” or “bulk separation.” Separation of molecules of similar sizes represents a fractionation that requires large resin beds. Generally, to achieve the required resolution to differentiate between molecules of similar sizes, the sample must be less than 5% the total volume of the resin bed volume. This type of fractionation is best achieved under pressure in systems such as FPLCs and HPLCs due to the required length of the column and associated back pressure.

Bulk separation, or buffer exchange, on the other hand requires much smaller rations of resin: sample, typically 3:1. As such, small volume samples can be efficiently desalted on a microscale. Such separations exist in both centrifugal device (e.g., Zeba Spin Desalting Plates/Columns) as well as microcolumns (e.g., Micro Bio-Spin™ Size Exclusion Spin Columns). Both devices utilize centrifugation.

Alternatively, pipette tips for automated liquid handlers (ALH) have been fashioned into size-exclusion columns. Iterations of such columns (tips) have been made commercially available by Biotage (née Phynexus, San Jose, CA) (see e.g., US Patent Publication 2009/0223893), IMCS (Irmo, SC), and DPX (Columbia, SC) (see e.g., US Patent Publication 2022/0184525). Tips from DPX swell the resin inside of the tip, while those from Biotage and IMCS use pre-swollen resin. Tips from DPX and Biotage rely on gravity flow of solutions through the swollen resin bed, while those from IMCS use positive pressure resulting from piston displacement to push liquid through the resin bed. As a result of dependence upon gravity flow, the durations of workflows for Biotage and DPX tips are roughly 30 minutes, whereas IMCStips report 10 to 15 minutes.

Previous examples of buffer exchange using IMCStips have been performed on the Hamilton Robotics platform of automated liquid handlers (ALH), which utilize a compressed O-ring to seal the pipette tips to the mandrels of the ALH. Several workflows incorporating SEC, such as multi-attribute method (Ogata et al. (2022) Rapid Comm. Mass Spec. 36: e9222, Sitasuwan et al. (2021) mAbs. 13:1978131), affinity protein purification, and low-endotoxin nucleic acid purification, have been demonstrated using the Hamilton Robotics ALH (Kates et al. (2023) SLAS technology. doi: 10.1016/j.slast.2023.01.005).

A key feature for using an SEC tip (such as IMCS's SizeX tip) on the Hamilton Robotics ALH is the use of Hamilton's CO-RE® tips. CO-RE® tips utilize a compressible o-ring that allows for a zero-pressure tip pick up. Because of this, tips can be positioned on the mandrel of either a pipetting channel or 96-pipetting head without applying pressure onto the liquid or resin bed. Zero pressure during this position step, where the tips are engaged with the mandrel of the ALH, is advantageous. Any pressure applied during tip engagement will force the sample through the resin, resulting in loss of sample or diluted sample. However, for Hamilton's CO-RE tips, zero pressure is applied at tip engagement and upon sealing with the compression O-ring, there is no subsequent breakthrough of the sample through the resin. At this point, specialty firmware commands can be employed to apply a specific amount of pressure as controlled by a defined plunger movement to obtain desalted biomolecules while retaining the salts in the resin.

Accordingly, automated microscale buffer exchange tips for use with ALHs utilizing a compressible o-ring that allows for zero-pressure tip pick up have provided improvements to both the speed of sample processing as well as reduction in manual intervention. There remains a need, however, for approaches to using automated microscale buffer exchange tips with other types of ALH systems that do not use such technology and that cannot afford to provide zero pressure fitting during pipette engagement with the mandrel.

### SUMMARY OF THE INVENTION

The disclosure provides kits and methods for using buffer exchange pipette tips and a pressure-fitted automated liquid handler (PF-ALH) for exchanging compounds, including large bio-molecules (e.g., proteins >20 kDa or large nucleic acids), into a buffer of interest. The methods for buffer exchange using SEC pipette tips and PF-ALH systems described herein comprise steps in which samples, and optionally buffers, are “top loaded” onto the SEC pipette tip (dispensed on top of the resin inside the pipette) using the PF-ALH system, and then a pressure differential is created (e.g., the plunger of the automated system is moved) prior to engaging the pipette tip to thereby facilitate movement of the sample or buffer through the resin, e.g., positive pressure is applied by air being pushed through the tip by the PF-ALH system which thereby facilitates movement of the sample or buffers through the tip to thereby accomplish buffer exchange via size exclusion. Modified workflows to accomplish top loading of the buffer exchange pipette tips, and creating a pressure differential prior to engagement of the pipette tips, using PF-ALH systems are described herein. Kits comprising buffer exchange pipette tips packaged with instructions for use with a PF-ALH system are also described herein.

Thus, this disclosure provides a rapid means to buffer exchange macromolecules without significant dilution while using a pressure-fitted automated liquid handler. In the kits and methods of the disclosure, the process for buffer exchange occurs completely inside the housing of the pipette tip. Moreover, the implementation of this method allows for parallel processing of up to 96 samples, greatly increasing the processing speed over traditional methods such as dialysis and gravity flow-based chromatography (approximately 10 minutes for up to 96 samples, as compared to 20 minutes for 8 samples via traditional gravity-flow chromatography). Furthermore, the kits and methods of the disclosure do not require additional hardware (centrifuges or liquid chromatography systems) and manual intervention is minimized. Accordingly, a rapid, PF-ALH-friendly SEC approach that allows for parallel processing and is not reliant on gravity represents a significant improvement in laboratory throughput and reliability.

In one aspect, the disclosure pertains to a method of buffer exchange for a sample, the method comprising:
providing a pressure-fitted automated liquid handler (PF-ALH) and a plurality of buffer exchange pipette tips situated in proximity to the PF-ALH,
wherein the buffer exchange pipette tips comprise a housing comprising (i) a proximal end comprising a lower opening; (ii) a distal end designed to fit the PF-ALH; and (iii) an inert resin for size exclusion chromatography (SEC) within the housing;
using the PF-ALH to dispense the sample on top of the inert resin inside the buffer exchange pipette tip;
using the PF-ALH to apply means for creating a pressure differential; and
using the PF-ALH to engage the distal end of the buffer exchange pipette tip to facilitate movement of the sample through the inert resin;
to thereby buffer exchange the sample.

In embodiments, the method further comprises, prior to dispensing the sample on top of the inert resin:
using the PF-ALH to dispense an equilibrium buffer on top of the inert resin in the buffer exchange pipette tip;
using the PF-ALH to apply means for creating a pressure differential; and
using the PF-ALH to engage the distal end of the buffer exchange pipette tip to facilitate movement of the equilibrium buffer through the inert resin.

In embodiments, the method further comprises, after movement of the sample through the inert resin:
using the PF-ALH to dispense an elution buffer on top of the inert resin in the buffer exchange pipette tip;
using the PL-ALH to apply means for creating a pressure differential; and
using the PF-ALH to engage the distal end of the buffer exchange pipette tip to facilitate movement of the elution buffer through the inert resin;
to thereby elute the sample from the inert resin into the elution buffer.

In embodiments, the PF-ALH comprises a plunger and the means for creating a pressure differential comprises moving the plunger prior to engaging the distal end of the buffer exchange pipette tip.

In embodiments, the inert resin is comprised of a base material selected from the group consisting of agarose, cellulose, dextran, polystyrene, polyacrylamide, polyacrylate, Sephadex, or Sepharose. In embodiments, the inert resin is Sephadex, Bio-gel P or Sepharose.

In embodiments, the housing of the pipette tip comprises a frit inside the housing positioned above the lower opening and the inert resin is positioned within the housing above the frit.

In embodiments, the buffer exchange pipette tip is a 1 ml tip. In embodiments, the buffer exchange pipette tip is a 5 ml tip. In embodiments, the buffer exchange pipette tip is an IMCStips® SizeX₁₀₀ tip, or an IMCStips® SizeX₁₅₀ tip, or an IMCStips® SizeX₇₅₀ tip, or an IMCStips® SizeX₁₀₀₀ tip.

In embodiments, the sample comprises a protein, such as a protein that is >10 kDa, >20 kDa, >50 kDa or >100 kDa.

In embodiments, the sample comprises a nucleic acid, such as plasmid DNA.

In embodiments, the sample comprises viral particles or lipid nanoparticles.

In embodiments, the PF-ALH is a Dynamic Devices® PF-ALH or a Tecan® PF-ALH.

In another aspect, the disclosure pertains to a kit for carrying out the methods of the disclosure. Accordingly, in embodiments, the disclosure provides a kit for buffer exchange comprising a plurality of buffer exchange pipette tips designed for use with a pressure-fitted liquid handler (PF-ALH) packaged with instructions for use of the buffer exchange pipette tips with the PF-ALH according to a method of the disclosure. Suitable tips, inert resins and PF-ALH systems for use in combination with the kits are as described herein.

While in no way intending to be limited to these specific embodiments, preferred parameters for use with particular PF-ALHs and buffer exchange pipette tips are described in detail herein, including in the Tables and Examples. For example, parameters for use of IMCStips® SizeX₁₀₀ tips or IMCStips® SizeX₁₅₀ tips with a Dynamic Devices® PF-ALH are provided in Table 1, parameters for use of IMCStips® SizeX₁₀₀ tips or IMCStips® SizeX₁₅₀ tips with a Tecan® PF-ALH are provided in Table 2 and parameters for use of IMCStips® SizeX₇₅₀ tips or IMCStips® SizeX₁₀₀₀ tips with a Dynamic Devices® PF-ALH are provided in Table 3.

### BRIEF DESCRIPTION OF THE DRAWINGS

FIG. 1 is a photograph of a representative example of a pipette tip for use in the kits and methods of the disclosure.

FIG. 2 is a bar graph showing results for protein (GFP) recovery across various initial concentrations when buffer exchanged using SizeXion tips on a Lynx LM1200 ALH system.

FIG. 3 is a bar graph showing results for protein (IgG) recovery across various initial concentrations when buffer exchanged using SizeXico tips on a Lynx LM1200 ALH system.

FIG. 4 is a bar graph showing results for protein (GFP) recovery across various initial concentrations when buffer exchanged using SizeX₁₅₀ tips on a Lynx LM1200 ALH system.

FIG. 5 is a bar graph showing results for protein (lgG) recovery across various initial concentrations when buffer exchanged using SizeX₁₅₀ tips on a Lynx LM1200 ALH system.

FIG. 6 is a bar graph showing results recovery of biomolecules of varying sizes when buffer exchanged using SizeX₁₀₀ tips on a Lynx LM1200 ALH system. CPMV: Cowpea mosaic virus, pDNA: plasmid DNA, IgG: Immunoglobulin G, BSA: bovine serum albumin, GFP: green fluorescent protein.

FIG. 7 is a bar graph showing results recovery of biomolecules of varying sizes when buffer exchanged using SizeX₁₅₀ tips on a Lynx LM1200 ALH system.

FIG. 8 is a bar graph showing results for protein (GFP) recovery across various initial concentrations when buffer exchanged using either SizeX₁₀₀ or SizeX₁₅₀ tips on a Tecan Fluent 1080 ALH system.

FIG. 9 is a chromatogram showing removal of a dye molecule when buffer exchanging using SizeX₁₀₀ tips on a Lynx LM1200 ALH system.

FIG. 10 is a chromatogram showing removal of a dye molecule while retaining sample when buffer exchanging using SizeX₁₀₀ tips on a Lynx LM1200 ALH system. The inset shows a more defined region of the chromatogram (475-550 nm).

FIG. 11 is a bar graph showing results for protein (GFP) recovery across various initial concentrations when buffer exchanged using SizeX₁₀₀₀ tips on a Lynx LM1200 ALH system.

FIG. 12 is a bar graph showing results for protein (IgG) recovery across various initial concentrations when buffer exchanged using SizeX₁₀₀₀ tips on a Lynx LM1200 ALH system.

FIG. 13 is a bar graph showing results for protein (GFP) recovery across various initial concentrations when buffer exchanged using SizeX₇₅₀ tips on a Lynx LM1200 ALH system.

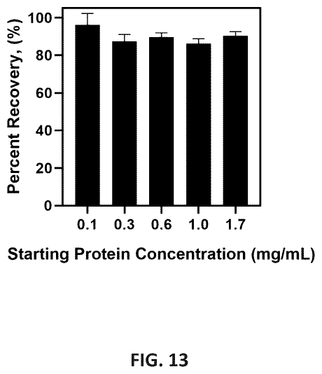

### DETAILED DESCRIPTION OF THE INVENTION

The invention provides an automated pipette-based system for exchanging macromolecules into a buffer of interest that involves using a pressure-fitted ALH (PF-ALH) system to first top-load buffer exchange pipette tips (designed to fit on the ALH system used) with appropriate buffers and samples and then to elute the samples in the buffer of interest. In the approach described herein for buffer exchange using a PF-ALH system, a pressure differential is created after the sample is dispensed but before the tip is engaged by the PF-ALH (e.g., the plunger of the automated system is moved prior to engaging the pipette tip) to thereby facilitate movement of liquid through the inert resin contained within the tips (e.g., by application of positive pressure to the tip). As described in the Examples, buffer exchange pipette tips suitable for various different sample volume sizes have been used with two different commercially-available pressure-fitted ALH systems to successfully buffer exchange a wide variety of substances, including small molecules (e.g., dyes) and biomolecules of varying sizes, including smaller proteins (<50 kDa) and larger proteins (>100 kDa), as well as large viral particles and plasmid DNA (e.g., ˜5 MDa).

Various aspects of the invention are described in further detail in the following subsections.

### I. Definitions

As used herein, the terms “top-load”, “top-loaded”, “top loading” and the like refer to the act of pipetting liquid into or onto the top of another pipette tip, in this case a tip containing resin for buffer exchange (a size exclusion chromatography tip).

As used herein, the term “tip pickup height” refers to a setting in the software of an ALH that determines the height at which the mandrels of the ALH channels or 96-head insert into the pipette tips. This setting does not mean the depth at which the mandrels have inserted into the tip.

As used herein, the term “equilibration” refers to the replacement of storage buffer with the buffer of interest.

As used herein, the term “sample dispense” refers to the placement of a set volume of sample on top of the swollen resin bed.

As used herein, the term “sample dispense volume” refers to either the volume or plunger positional displacement applied to the tip/sample after the sample dispense step and following tip pickup.

As used herein, the term “hold time” refers to either the time after tip pick up or the amount of time after the piston movement is complete that a tip is held in position before either being moved to the tip rack or dispensed from the ALH.

As used herein, the term “chaser dispense” refers to the placement of a set volume of buffer on top of the swollen resin bed.

As used herein, the term “chaser dispense volume” refers to either the volume or plunger positional displacement applied to the tip/sample after the chaser dispense step and following tip pickup.

### II. Automated Liquid Handling Systems

The kits and methods of the disclosure are used in conjunction with an Automated Liquid Handler (ALH) in which the pipette tips are pressure-fitted onto the mandrel(s) of the ALH, referred to herein as a pressure-fitted ALH system or PF-ALH system. This is in contrast to ALH systems that use an O-ring to fit the pipette tip onto the mandrels, such as the Hamilton Robotics automated liquid handling platforms, referred to herein as O-ring-based ALH systems or OR-ALH. In OR-ALH systems, the pipette tips can be positioned onto the mandrel of either a pipetting channel or 96-pipetting head without applying pressure onto the liquid or resin bed within the tip, whereas in PF-ALH systems, positioning of the pipette tips onto the mandrel(s) applies pressure to the liquid or resin bed within the tip.

Numerous PF-ALH systems are commercially available in the art. In one embodiment, the PF-ALH system is a Dynamic Devices ALH, such as a Lynx platform ALH (e.g., Lynx LM1200) that utilizes Volume Verified Pipetting (VVP). In another embodiment, the PF-ALH system is a Tecan ALH, such as the Fluent platform (e.g., Fluent 1080). In yet other embodiments, the PF-ALH system is a Tecan EVO ALH, an Analytik Jena ALH, an Eppendorf ALH or an Opentrons ALH, which each utilize pressure-fit tip pickup.

### III. Buffer Exchange Pipette Tips

The kits and methods of the disclosure utilize a buffer exchange pipette tip. The buffer exchange pipette tip is designed or adapted to fit on the particular ALH to be used for buffer exchange. For example, when a Dynamic Devices ALH, such as a Lynx platform ALH, is used, the buffer exchange pipette tip is designed to fit on this ALH. In certain instances, the buffer exchange pipette tip is prepared from empty pipette tips from the ALH manufacturer that are designed for use with that ALH.

The buffer exchange pipette tip comprises an inert resin, porous in nature, that facilitates buffer exchange through size exclusion chromatography. Typically, the inert resin is comprised of a base material such as agarose, cellulose, dextran, polystyrene, polyacrylamide, polyacrylate, Sephadex, or Sepharose. Non-limiting examples of commercially available inert resins include: Sephadex, (cross-linked dextran epichlorohydrin), Bio-gel P (polyacrylamide), and Sepharose (agarose). In embodiments, the resin is pre-swollen within the pipette tip.

Typically, a pipette tip comprises inert resin in an amount from 150 mg to 500 mg in a 1000 μL (1 ml) tip. In embodiments, the pipette tip is a 1 ml tip. In other embodiments, the pipette tip is a 5 ml tip.

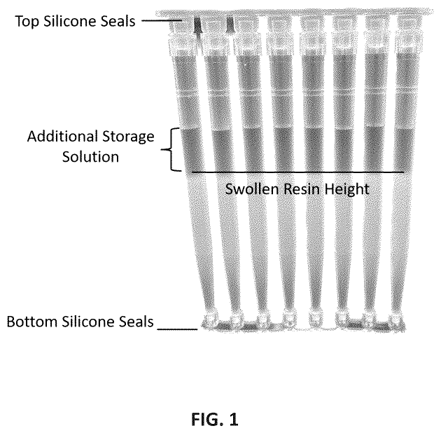

A non-limiting embodiment of the buffer exchange pipette tip is shown in FIG. 1, which illustrates how the inert resin in positioned within the chamber of the pipette tip. The height of the swollen resin within the tip is indicated, as well as that of the additional storage solution. The top and bottom of the pipette tips are closed with a silicone seal for storage.

In embodiments, the pipette tip for buffer exchange comprises:
a housing having a proximal end containing a lower opening and a distal end designed to fit a pipette mandrel (e.g., an individual channel or the multi-channel pipetting head of an ALH), optionally wherein the taper angle formed between the proximal end and distal end is 10, 9, 8, 7, 6 or 5 degrees or fewer;
a frit, mesh or screen inside the housing positioned above the lower opening; and
a plurality of pre-swollen inert particles comprised of a crosslinked polymer positioned within the housing above the frit.

In embodiments, the inert particles are selected from the group consisting of agarose, cellulose, dextran, polystyrene, polyacrylamide, polyacrylate, Sephadex, or Sepharose.

In embodiments, the buffer exchange pipette tip is an IMCStips® size exclusion tip, commercially available from IMCS (Irmo, SC). Non-limiting examples include SizeX₁₀₀ tips, SizeX₁₅₀ tips, SizeX₇₅₀ tips and SizeX₁₀₀₀ tips, each of which use a 1 ml (1000 μL) tip containing a size exclusion resin and which are suitable for 100 μl, 150 μl, 750 μl or 1000 μl sample volumes, respectively. The SizeX₁₀₀ tips and SizeX₁₅₀ tips are 1 ml tips. The SizeX₇₅₀ tips and SizeX₁₀₀₀ tips are 5 ml tips.

In embodiments, the buffer exchange pipette tip is arranged (e.g., packaged) into a plurality of tips (e.g., 8 tips or 96 tips).

In embodiments, the buffer exchange pipette tip is further modified to include a thin (<2 mm) barrier, filter, or screen placed on top of the resin bed (e.g., pre-swollen resin bed), for example to help maintain resin position and packing during shipping.

### IV Pipette Tip Preparation

Pre-swollen resin-containing tips (e.g., SizeX tips) typically are packaged with both proximal and distal ends plugged with seals comprised of silicone or some derivative thereof, as illustrated in FIG. 1. DPX promotes dry resin and swelling the resin prior to its use. However, not using pre-swollen resin can diminish recovery of biomolecules during buffer exchange. Removal of the top seals is followed by removal of the bottom seals, yielding a size exclusion chromatography pipette tip for use in the methods of buffer exchange of the disclosure. Tips are stored in a storage buffer containing a preservative solution that is removed prior to use. Prior to storage buffer removal, the resin in the tips is settled, such as by flicking with a quick wrist motion to settle any resin jostled during shipping.

Storage buffer removal can be accomplished in various ways, such as by gravity flow, centrifugation at 200×g for 1 minute, or by using positive pressure delivered by the ALH after tip pickup. If the tips are drained of storage buffer by gravity flow or by centrifugation, tips are then placed in a standard tip rack on the ALH deck for use with the ALH system.

### V. Buffer Exchange Workflow

The general workflow used for buffer exchange is to equilibrate the column in the buffer the user wants the final product to be in, load the sample of interest, and elute the material off with a set amount of buffer. The goal is to use the length of the column and the diffusion of smaller molecules into the pores of the resin to separate the analyte of interest from molecules of different sizes present in the original sample.

The workflow for this disclosure follows that of traditional size-exclusion column chromatography. The workflow involves loading the sample onto the inert resin in the tip and passing the sample through the resin and, optionally, can also include an equilibration step prior to sample loading, and/or an elution step after sample loading. The workflow of the disclosure is adapted for use with buffer exchange pipette tips and PF-ALHs such that (i) the buffers and samples are loaded on top of the resin bed inside the pipette tips (top-loaded) for each step using the automated system; and (ii) means are applied to create a pressure differential (e.g., the plunger of the automated system is moved) prior to engaging to the pipette tip at each step of the buffer exchange process and positive pressure is applied to facilitate the movement of liquid through the tip. This contrasts with the standard use of ALHs in which the pipette tip is first engaged by the automated system and then a pressure differential is applied (e.g., the plunger of the automated system is moved) to facilitate movement of liquid.

Accordingly, in one aspect, the disclosure provides a method of buffer exchange for a sample, the method comprising:
providing a pressure-fitted automated liquid handler (PF-ALH) and a plurality of buffer exchange pipette tips situated in proximity to the PF-ALH,
wherein the buffer exchange pipette tips comprise a housing comprising (i) a proximal end comprising a lower opening; (ii) a distal end designed to fit the PF-ALH; and (iii) an inert resin for size exclusion chromatography (SEC) within the housing;
using the PF-ALH to dispense the sample on top of the inert resin inside the buffer exchange pipette tip;
using the PF-ALH to apply means for creating a pressure differential; and
using the PF-ALH to engage the distal end of the buffer exchange pipette tip to facilitate movement of the sample through the inert resin;
to thereby buffer exchange the sample.

In embodiments, the method further comprises, prior to dispensing the sample on top of the inert resin:
using the PF-ALH to dispense an equilibrium buffer on top of the inert resin in the buffer exchange pipette tip;
using the PF-ALH to apply means for creating a pressure differential, and
using the PF-ALH to engage the distal end of the buffer exchange pipette tip to facilitate movement of the equilibrium buffer through the inert resin.

In embodiments, the method further comprises, after movement of the sample through the inert resin:
using the PF-ALH to dispense an elution buffer on top of the inert resin in the buffer exchange pipette tip;
using the PL-ALH to apply means for creating a pressure differential; and
using the PF-ALH to engage the distal end of the buffer exchange pipette tip to facilitate movement of the elution buffer through the inert resin;
to thereby elute the sample from the inert resin into the elution buffer.

In embodiments, the PF-ALH comprises a plunger and the means for creating a pressure differential comprises moving the plunger prior to engaging the distal end of the buffer exchange pipette tip. For example, the PF-ALH can be one that employs air displacement pipetting, which relies on an air cushion to move liquid through the pipette tip. Accordingly, in embodiments, the PF-ALH comprises a plunger and employs air displacement to move liquid (e.g., the sample or buffer) through the resin situated inside the housing of the pipette tip.

Alternatively, automated liquid handlers that employ other types of displacement technologies are known in the art and can be adapted to the methods of the disclosure. For example, in another embodiment, the PF-ALH employs positive displacement pipetting, which involves direct contact between a liquid (e.g., the sample or buffer) and a disposable piston or tip, wherein movement of the piston physically displaces the liquid. Accordingly, in another embodiment, the PF-ALH comprises a piston or tip (e.g., disposable piston or tip) and employs positive displacement to move the liquid (e.g., the sample or buffer) through the resin situated inside the housing of the pipette tip.

In the PF-ALH systems known in the art, software is used to carry out the workflow, including determining the pipetting and tip pick-up locations, the amount of volume of liquid dispensed, the creation of a pressure differential, the amount of air dispensed and other parameters.

To carry out the top-loading steps of the method of the disclosure, the PF-ALH software is manipulated to accomplish top-loading by adjusting the pipetting and tip pick-up locations to alter between plates and tip racks, respectively. The software is manipulated to identify the liquid (buffer or sample) being top loaded onto the pipette tip as if samples are dispensed into a plate, not tips. However, such a plate is virtually present and not physically present on the ALH. After top-loading, the software is programmed to identify the deck position as a tip rack, allowing for tip pick up of the loaded SEC tip. This switching between tip and a plate in the software is one of the key steps in ensuring proper execution of this workflow.

Depending on the PF-ALH system used, this task may be performed differently. For example, using a Dynamic Devices PF-ALH system (e.g., Lynx) and software, this is performed via a developed script that flips plate and tip rack positions on the fly. This is added as an individual step after either tip piston displacement or prior to tip pick up.

Using a Tecan PF-ALH system (e.g., Fluent) and software, a 96-well plate can be moved from a deck position to the top of the tip rack via the “Set Location” command. After top loading is complete, the 96-well plate can be set back to its designated deck position via the same “Set Location” command enabling tip pick up.

A second software-based challenge is the movement of the piston prior to picking up pipette tips. In general, this is prevented by ALH pipetting as a safeguard. In the workflow of this disclosure, displacement of the piston to simulate preaspiration can be accomplished in different ways depending on the PF-ALH system used.

For example, on the Dynamics Devices software, aspiration of a defined volume of air (preaspiration) can be performed by first using the “Set Tip Type” command to have tips present on the head. The aspiration command then follows, and then another “Set Tip Type” command is set to “none.” At this point, there have been no actual tips picked up by the system, but the plungers controlling pipetting have been positioned to allow a defined volume to be dispensed upon tip pick up. The tip is then picked up in a normal fashion and dispense volumes related to the sample dispense volume or chaser dispense volume can be applied.

On the Tecan Fluent software, the “Prepare Axis Move” command is used with similar functionality. The “Prepare Axis Move” command allows for manual control of plungers on the Flexible Channel Arm (FCA). In this command, the plunger movement is controlled by a distance (position in) mm/° rather than volume. The speed of this plunger movement is designated by a percentage, or factor, of the positional displacement. After each of the plungers is prepared, tips are picked up in a normal fashion. The “Prepare Axis Move” is then utilized again to restore the position to 0 mm. In embodiments, a low factor (e.g., between 0-20%) allows for slow depression of the plunger and a controlled push of liquid into the resin bed.

These two software solutions are used in each of the three steps of size exclusion chromatography: equilibration, sample load, and elution. Non-limiting examples of optimal parameters for IMCStips SizeX₁₀₀ and SizeX₁₅₀ tips on both the Dynamic Devices Lynx LM1200 and Tecan Fluent 1080 are summarized below in Table 1 and Table 2, respectively.

TABLE 1
Optimized parameters for DDSizeX₁₀₀ and
DDSizeX₁₅₀ on a Dynamic Devices Lynx LM1200.

| | DDSizeX₁₀₀ Parameters | | | DDSizeX₁₅₀ Parameters | | |
|---|---|---|---|---|---|---|
| SVP | Volume, | Plunger | Hold | Volume, | Plunger | Hold |
| Step | μL | Dispense, μL | Time, s | μL | Dispense, μL | Time, s |
| Equilibration | 300 | 750 | 20 | 300 | 750 | 20 |
| Equilibration | 300 | 750 | 20 | 300 | 750 | 20 |
| Equilibration | 300 | 750 | 20 | 300 | 750 | 20 |
| Sample | 100 | 0 | 3 | 150 | 50 | 20 |
| Chaser | 100 | 125 | 30 | 150 | 150 | 60 |

TABLE 2
Optimized parameters for SizeX₁₀₀ and SizeX₁₅₀ on the Tecan Fluent.

| | Tecan SizeX₁₀₀ | | | | Tecan SizeX₁₅₀ | | | |
|---|---|---|---|---|---|---|---|---|
| | | | | Hold | | | | Hold |
| | Volume | Position | Factor | Time, | Volume | Position | Factor | Time, |
| Step | (μL) | [mm/°] | [%] | s | (μL) | [mm/°] | [%] | s |
| Blowout | — | 50 | 100 | 3 | — | 50 | 100 | 3 |
| Equilibration | 450 | 120 | 10 | 25 | 450 | 120 | 10 | 25 |
| Equilibration | 450 | 120 | 10 | 25 | 450 | 120 | 10 | 25 |
| Sample | 100 | 10 | 10 | 12 | 150 | 25 | 10 | 12 |
| Chaser | 100 | 15 | 5 | 15 | 150 | 27 | 5 | 15 |

Non-limiting examples of optimal parameters for IMCStips SizeX₇₅₀ and SizeX₁₀₀₀ tips on the Dynamic Devices Lynx LM1200 are summarized below in Table 3.

TABLE 3
Optimized parameters for DDSizeX₇₅₀ and
DDSizeX₁₀₀₀ on a Dynamic Devices Lynx LM1200.

| | DDSizeX₁₀₀₀ Parameters | | DDSizeX₇₅₀ Parameters | | | |
|---|---|---|---|---|---|---|
| | | Plunger | | | Plunger | |
| SVP | Volume, | Dispense, | Hold | Volume, | Dispense, | Hold |
| Step | μL | μL | Time, s | μL | μL | Time, s |
| Equilibration | 1300 | 2500 | 12 | 1750 | 3500 | 0 |
| Equilibration | 1300 | 2500 | 12 | 1750 | 3500 | 0 |
| Equilibration | 1300 | 2500 | 12 | — | — | — |
| Equilibration | 1300 | 2500 | 12 | — | — | — |
| Sample | 1000 | 350 | 60 | 750 | 150 | 15 |
| Chaser | 1050 | 1050 | 60 | 750 | 800 | 40 |

The parameters for buffer exchange on different PF-ALH systems were optimized for equilibration, sample load, and elution steps as follows.

For equilibration, the total volume of equilibration buffer was tested between 300-5200 μL. For both the 1 mL Dynamic Devices and Tecan tips, 900 μL of equilibration buffer was sufficient to remove the storage buffer. In the case of the Dynamic Devices tips, three successive cycles of 300 μL of equilibration buffer followed by a 750 μL plunger dispense was optimal. Each plunger dispense was followed by a 20 second hold time. Equilibration solution was dispensed into a waste trough and returned to the tip pick-up position prior to each iteration. Both SizeX₁₀₀ and SizeX₁₅₀ tips were able to be fully equilibrated in the same manner. For the Tecan tips, a similar workflow was developed. In this case, there were only two successive cycles of 450 μL equilibration buffer followed by plunger dispense. The settings for the plunger position were 120 for both SizeX₁₀₀ and SizeX₁₅₀ tips with a 25 second hold time in between steps. After equilibration, both tips contained resin that was equilibrated with the desired final buffer for the analyte of interest. For the larger volume (5 mL) tips, between 3000-5500 μL of equilibration buffer was tested. 3500 μL of equilibration buffer was sufficient to remove storage buffer for the SizeX₇₅₀ tips and 5200 μL of equilibration buffer was sufficient to remove storage buffer for the SizeX₁₀₀₀ tips. Equilibrations were performed between two and four cycles. The larger resin bed of the SizeX₁₀₀₀ tips left less room for equilibration buffer to be added, and an upper limit of 1350 μL per bolus was determined.

For the sample load step, to the now equilibrated resin, an aqueous sample was then added. The volume of sample typically is from 1-200 μL with the optimal volumes for SizeX₁₀₀ tips found to be 100 μL and the optimal value for SizeX₁₅₀ tips to be 150 μL. Ideally, this sample is loaded as low to the resin bed as possible so as to minimize disruption of the bed. For the Dynamic Devices PF-ALH system, the sample dispense step was accomplished with a 1250 μL tip attached to the 96-head, but could also be performed with 230 μL and 340 μL tips. For the Tecan PF-ALH system, sample dispense was performed with 1 mL tips, but this could also be performed with 200 μL and 350 μL tips.

After the sample is loaded on top of the resin bed, the sample is depressed into the resin bed during a tip pick-up step that may or may not contain additional plunger dispense volume, depending on the PF-ALH system used. For example, for SizeX₁₀₀ on the Dynamic Devices PF-ALH system (DDS), there was no additional plunger dispense needed and a three-second hold time was used. For SizeX₁₅₀ on the DDS, there was an additional 50 μL plunger dispense followed by a 20-second hold time. The difference in sample volume from sample dispense volume suggests that the insertion of the mandrel into the tip at a depth of 11 mm equated to roughly 100 μL of displaced air. This represents a significant difference between prior approaches and this disclosure and is responsible for the ability to apply positive pressure to the system and not rely on gravity flow.

Identification of the required mandrel depth to retain tip contact as well as accommodation and adjustments of volumes for downstream steps was not previously required using O-ring-based ALH systems, such as Hamilton Robotics systems. Indeed, insufficient resin bed heights coupled with volume displacement from a pressure-fit tip pick up would greatly decrease yield due to prematurely pushing sample out of the tip. Moreover, insufficient mandrel depth with result in the tip being ejected during plunger dispense due to insufficient tip-mandrel contact for the built-up backpressure. On the Tecan system, the positional displacements for SizeX₁₀₀ and SizeX₁₅₀ sample dispense volumes are 10 μL and 25 μL, respectively. Additional hold times are 12 seconds for both tip types. Values significantly higher than 10 mm resulted in significant sample loss during the loading step for SizeX₁₀₀. After the sample dispense volume is added, the sample is retained in the resin bed and the tip is replaced in the tip pick-up position.

For the elution step, an aqueous chaser composed of the same buffer as the equilibration buffer is then added to the top of the resin bed via top loading. For SizeX₁₀₀ and SizeX₁₅₀, the volumes of buffer added were 100 μL and 150 μL regardless of the system. On the Dynamic Devices system, various plunger dispense volumes were tested (50-250 μL) with optimal values determined to be 125 and 150 μL for SizeX₁₀₀ and SizeX₁₅₀, respectively. On the Tecan system, various plunger dispense distance were tested (10-50 mm) with optimal values determined to be 15 and 27 mm for SizeX₁₀₀ and SizeX₁₅₀, respectively.

### VI. Kits

In another aspect, the disclosure provides kits for performing the methods of the disclosure. In embodiments, the kits comprise a plurality of buffer exchange pipette tips designed for use with a PF-ALH, in a package with instructions for using the buffer exchange pipette tips with the PF-ALH for exchanging a sample into a buffer of interest.

The buffer exchange pipette tips included in the kits are described above in Subsection III. In embodiments, the buffer exchange pipette tips are 1 ml tips. In embodiments, the buffer exchange pipette tips are 5 ml tips. In embodiments, the tips are IMCStips for size exclusion chromatography, such as SizeX₁₀₀, SizeX₁₅₀, SizeX₇₅₀, or SizeX₁₀₀₀ tips.

Automated liquid handlers are described above in Subsection II. In an embodiment, the buffer exchange pipette tips and instructions for use are designed for use with a Dynamic Devices PF-ALH, such as a Lynx platform. In an embodiment, the buffer exchange pipette tips and instructions for use are designed for use with a Tecan PF-ALH, such as a Fluent platform.

Workflows for using buffer exchange pipette tips with different PF-ALH systems are described above in Subsection V. The instructions included in the kit comprise directions for carrying out the workflow, using the tips and the PF-ALH system of interest, the buffer-exchange a sample into a buffer of interest.

### Examples

### Example 1: Small Molecule Removal Efficiency Using SizeX₁₀₀ Tips on a Dynamic Devices Automated Liquid Handling System

In this example, a UV-vis spectrometer was used to measure the absorbance of a solution containing a small molecule (dye) prior to and after being passed through a SizeX₁₀₀ tip using the multi-channel head on a Dynamic Devices Lynx LM1200.

The base of the SizeX₁₀₀ tips were constructed by placing a porous 0.072″ polyethylene frit at 3.5 mm above the apical orifice of a 1200 μL wide bore tip (Dynamic Devices, Arizona). 262.5 mg (wet mass) of an inert resin with a 1,000-6,000 MW fractionation range (Bio-Gel P-6DG) was added to the top of the frit and allowed to settle. Tips were capped on both ends and stored at 4° C. until they were ready for use.

Tips were prepared for use in the method in either of two manners. Prior to use on the Lynx system, tips were centrifuged at 200×g for 1 minute. Alternatively, tips were placed directly in a tip rack. The plungers on the multi-channel heads were pulled up to a height corresponding to 750 μL and the SizeX₁₀₀ tips were then picked up by the multi-channel head and the plungers fully dispensed. The resulting action of both methods was a packed bed and the removal of storage solution.

The general workflow used for buffer exchange is to equilibrate the column in the buffer the user wants the final product to be in, load the sample of interest, and elute the material off with a set amount of buffer. The goal is to use the length of the column and the diffusion of smaller molecules into the pores of the resin to separate the analyte of interest from molecules of different sizes present in the original sample.

In this example, a small molecule detectable by absorbance, tartrazine, was used to determine what percent of a small molecule could be removed from a starting sample. The absorbance of tartrazine, a yellow dye, was measured at 425 nm on a NanoDrop 2000. A standard curve of tartrazine (0.015-1.00 mM) was constructed using the NanoDrop 2000. Two concentrations of tartrazine, 20 mM and 100 mM were used as sample inputs (100 μL) onto the top of a SizeX₁₀₀ column bed that had been equilibrated with 900 μL of deionized water. In this example, there was no additional piston displacement after tips were picked up. Tips were picked up at a height of 4.5 relative to the deck position, resulting in the insertion of the mandrel of the pipetting head at least 11 mm into the tops of the SizeX₁₀₀ tips. As the pipetting head was inserted into the SizeX₁₀₀ tips, the tartrazine sample was pushed into the resin bed and liquid was displaced out of the apical ends of the SizeX₁₀₀ tips. Tips were then placed back into the tip rack. 100 μL of chaser solution (deionized water) was added on top of the resin bed. The plunger on the multi-channel pipetting head was extended to a volume of 125 μL prior to tip pick up. Tips were then picked up and positioned over a collection plate. The plungers were dispensed and tips were allowed to drain for 30 additional seconds. The eluted material was then diluted by a factor of twenty-fold and the absorbance was measured at 425 nm. The concentration of tartrazine in the eluted material was calculated by translating the measured absorbance value at 425 nm to concentration via a standard curve and then multiplying by the dilution factor. The measured remaining tartrazine concentrations for the 100 mM and 20 mM samples were 1.87±0.71 mM (11=3) and 0.43±0.06 mM (n=3), respectively. This equates to >98% small molecule removal over both concentrations tested.

### Example 2: Small Molecule Removal Efficiency Using SizeX₁₅₀ Tips on a Dynamic Devices Automated Liquid Handling System

In this example, a UV-vis spectrometer was used to measure the absorbance of a solution containing a small molecule (dye) prior to and after being passed through a SizeX₁₅₀ tip using the multi-channel head on a Dynamic Devices Lynx LM1200.

The base of the SizeX₁₅₀ tips were constructed by placing a porous 0.072″ polyethylene frit at 3.5 mm above the apical orifice of a 1200 μL wide bore tip (Dynamic Devices, Arizona). 350 mg (wet mass) of an inert resin with a 1,000-6,000 MW fractionation range (Bio-Gel P-6DG) was added to the top of the frit and allowed to settle. Tips were capped on both ends and stored at 4° C. until they were ready for use.

Tips could be prepared for use in the method in two manners. Prior to use on the Lynx system, tips could be centrifuged at 200×g for 1 minute. Alternatively, tips could be placed directly in a tip a rack. The plungers on the multi-channel heads could be pulled up to a height corresponding to 750 μL and the SizeX₁₅₀ tips could then be picked up by the multi-channel head and the plungers fully dispensed. The resulting action of both methods was a packed bed and the removal of storage solution.

The general workflow used for buffer exchange is to equilibrate the column in the buffer the user wants the final product to be in, load the sample of interest, and elute the material off with a set amount of buffer. The goal is to use the length of the column and the diffusion of smaller molecules into the pores of the resin to separate the analyte of interest from molecules of different sizes present in the original sample.

In this example, a small molecule detectable by absorbance, tartrazine, was used to determine what percent of a small molecule could be removed from a starting sample. The absorbance of tartrazine, a yellow dye, was measured at 425 nm on a NanoDrop 2000. A standard curve of tartrazine (0.015-1.00 mM) was constructed using the NanoDrop 2000. Two concentrations of tartrazine, 20 mM and 100 mM were used as sample inputs (150 μL) onto the top of a SizeX₁₅₀ column bed that had been equilibrated with 900 μL of deionized water. In this example, there was a piston displacement equivalent to 50 μL of volume prior to the tips being picked up. Tips were picked up at a height of 4.5 relative to the deck position, resulting in the insertion of the mandrel of the pipetting head at least 11 mm into the tops of the SizeX₁₅₀ tips. As the pipetting head was inserted into the SizeX₁₅₀ tips, the tartrazine sample was pushed into the resin bed and liquid was displaced out of the apical ends of the SizeX₁₅₀ tips. The plunger was then dispensed and the tips were allowed to drain for an additional twenty seconds. Tips were then placed back into the tip rack. 150 μL of chaser solution (deionized water) was added on top of the resin bed. The plunger on the multi-channel pipetting head was extended to a volume of 150 μL prior to tip pick up. Tips were then picked up and the positioned over a collection plate. The plungers were dispensed and tips were allowed to drain for 60 additional seconds. The eluted material was then diluted by a factor of twenty-fold and the absorbance was measured at 425 nm. The concentration of tartrazine in the eluted material was calculated by translating the measured absorbance value at 425 nm to concentration via a standard curve and then multiplying by the dilution factor. The measured remaining tartrazine concentrations for the 100 mM and 20 mM samples were 1.18±0.21 mM (n=3) and 0.43±0.08 mM (n=3), respectively. This equates to >98% small molecule removal over both concentrations tested.

### Example 3: Recovery of Small Protein (<50 kDa) Samples Using SizeX₁₀₀ Tips on a Dynamic Devices Automated Liquid Handling System

An important characteristic of a buffer exchange device is to recover a high percentage of the analyte of interest without excessive dilution. In this example, a recombinant protein, his₆-tagged green fluorescent protein (GFP) (MW: 27,402 Da), was used to demonstrate a small protein could be buffer exchanged while maintaining a similar concentration at high yield using SizeX₁₀₀ tips on a Dynamic Devices Lynx LM1200. Methodology for measuring the recovery of GFP was similar to that described in Example 1 with a few exceptions. In this example, the material used as a sample load was 100 μL of 0.958 mg/mL GFP in 1x PBS. Initial concentration of GFP was determined by NanoDrop 2000 using a calculated extinction coefficient of 25102 M⁻¹ cm⁻¹ according to the methodology described by Gill and von Hippel (Analytical Biochemistry, 1989, 182 (2), 319-326). As in Example 1, there was no additional plunger dispense after the sample was pushed into the resin bed by the insertion of the mandrel in the SizeX₁₀₀ tip. Material was eluted from the SizeX₁₀₀ column as described in Example 1. The volume of the eluted material was measured by a manual Eppendorf 200 μL pipette. The concentration of the eluted material was measured at 280 nm on a NanoDrop2000. The average (n=4) eluted material was 103±3.74 μL with a concentration of 0.765±0.033 mg/mL resulting in an average yield of 82.21:0.97%. These results correspond to a 25% dilution of the initial analyte solution.

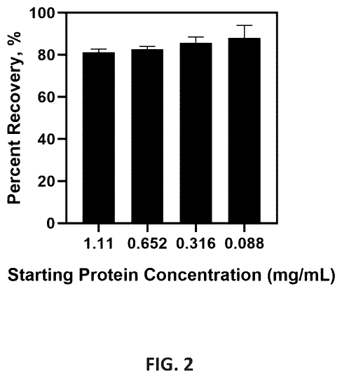

In a related example, it was demonstrated that the initial concentration of the sample analyte had little bearing on the yield or dilution factor. In this example, 100 μL of GFP at four initial concentrations (1.11, 0.652, 0.316, and 0.088 mg/mL) was used as the sample of interest. The buffer exchange protocol followed as was described earlier in Example 3. Yields, as shown in FIG. 2, (n=4) were 81.0±1.24%, 82.6±1.13%, 85.9±2.28%, and 88.0±5.25% for 1.11, 0.652, 0.316, and 0.088 mg/mL, respectively. Eluted volumes were 102±2.22, 103±2.22, 104±3.32, and 104±2.89 μL for the same concentrations, respectively. The final concentrations (and dilutions) for each of the four concentrations were 0.858±0.022 mg/mL (30%), 0.527±0.010 mg/mL (23%), 0.259±0 011 mg/mL (22%), and 0.075±0.003 mg/mL (18%), respectively.

### Example 4: Recovery of Small Protein (>100 kDa) Samples Using SizeX₁₀₀ Tips on a Dynamic Devices Automated Liquid Handling System

In this example, a polyclonal human IgG (IgG) (Immunoreagents Inc., Raleigh, NC) (MW˜150 kDa), was used to demonstrate large proteins could be buffer exchanged while maintaining a similar concentration at high yield using SizeX₁₀₀ tips on a Dynamic Devices Lynx LM1200. Methodology for measuring the recovery of IgG was similar to that described in Example 1 with a few exceptions. In this example, the material used as a sample load was 100 μL of 1.029 mg/mL IgG in 1x PBS. Initial concentration of IgG was determined by NanoDrop 2000 using an E1% of 13.7. As in Example 1, there was no additional plunger dispense after the sample was pushed into the resin bed by the insertion of the mandrel in the SizeX₁₀₀ tip. Material was eluted from the SizeX₁₀₀ column as described in Example 1. The volume of the eluted material was measured by a manual Eppendorf 200 μL pipette. The concentration of the eluted material was measured at 280 nm on a NanoDrop2000. The average (n=4) eluted material was 99.8±1.50 μL with a concentration of 0.848±0.030 mg/mL resulting in an average yield of 82.1±1.72%. These results correspond to a 21% dilution of the initial analyte solution.

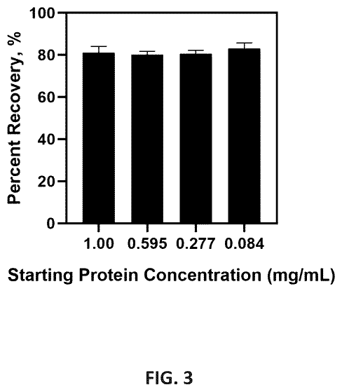

In a related example, it was demonstrated that the initial concentration of a large protein sample analyte had little bearing on the yield or dilution factor. In this example, 100 μL of IgG at four initial concentrations (1.00, 0.595, 0.277, and 0.084 mg/mL) was used as the sample of interest. The buffer exchange protocol followed as was described earlier in Example 4. Yields, as shown in FIG. 3, (n=4) were 80.8±2.93%, 80.3±1.44%, 80.5±1.27%, and 83.1±2.40% for 1.00, 0.595, 0.277, and 0.084 mg/mL, respectively. Eluted volumes were 99.8±2.62, 102±3.32, 105±2.22, and 101±1.26 AL for the same concentrations, respectively. The final concentrations (and dilutions) for each of the four concentrations were 0.813±0.029 mg/mL (23%), 0.471±0.015 mg/mL (26%), 0.213±0.008 mg/mL (30%), and 0.069±0.002 mg/mL (21%), respectively.

### Example 5: Recovery of Small Protein (<50 kDa) Samples Using SizeX₁₅₀ Tips on a Dynamic Devices Automated Liquid Handling System

An important characteristic of a buffer exchange device is to recover a high percentage of the analyte of interest without excessive dilution. In this example, a recombinant protein, his₆-tagged green fluorescent protein (GFP) (MW: 27,402 Da), was used to demonstrate a small protein could be buffer exchanged while maintaining a similar concentration at high yield using SizeX₁₅₀ tips on a Dynamic Devices Lynx LM1200. Methodology for measuring the recovery of GFP was similar to that described in Example 2 with a few exceptions. In this example, the material used as a sample load was 150 μL of 0.918 mg/mL GFP in 1x PBS. Initial concentration of GFP was determined by NanoDrop 2000 using a calculated extinction coefficient of 25102 M-1 cm 1 according to the methodology described by Gill and von Hippel (Analytical Biochemistry, 1989, 182 (2), 319-326). As in Example 2, there was an additional plunger dispense of 50 μL and hold time of twenty seconds after the sample was pushed into the resin bed by the insertion of the mandrel in the SizeX₁₅₀ tip. Material was eluted from the SizeX₁₅₀ column as described in Example 2. The volume of the eluted material was measured by a manual Eppendorf 200 μL pipette. The concentration of the eluted material was measured at 280 nm on a NanoDrop2000. The average (n=4) eluted material was 153=2.16 μL with a concentration of 0.773±0.018 mg/mL resulting in an average yield of 85.8±2 73%. These results correspond to a 19% dilution of the initial analyte solution.

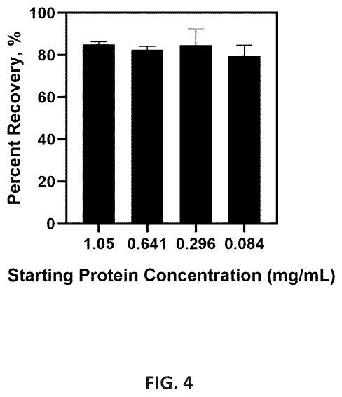

In a related example, it was demonstrated that the initial concentration of the sample analyte had little bearing on the yield or dilution factor. In this example, 150 μL of GFP at four initial concentrations (1.05, 0.641, 0.296, and 0.084 mg/mL) was used as the sample of interest. The buffer exchange protocol followed as was described earlier in Example 5. Yields, as shown in FIG. 4, (n=4) were 85.0±1.56%, 82.4±1.38%, 84.7±6.46%, and 79.5±4 34% for 1.05, 0.641, 0.296, and 0.084 mg/mL, respectively. Eluted volumes were 144±3.59, 146±2.83, 147±2.36, and 149±1.41 μL for the same concentrations, respectively. The final concentrations (and dilutions) for each of the four concentrations were 0.929±0.038 mg/mL (13%), 0.543±0.015 mg/mL (18%), 0.256=0.019 mg/mL (16%), and 0.067±0.004 mg/mL (25%), respectively.

### Example 6: Recovery of Small Protein (>100 kDa) Samples Using SizeX₁₅₀ Tips on a Dynamic Devices Automated Liquid Handling System

In this example, a polyclonal human IgG (IgG) (Immunoreagents Inc., Raleigh, NC) (MW˜150 kDa), was used to demonstrate large proteins could be buffer exchanged while maintaining a similar concentration at high yield using SizeX₁₅₀ tips on a Dynamic Devices Lynx LM1200. Methodology for measuring the recovery of IgG was like that described in Example 1 with a few exceptions. In this example, the material used as a sample load was 150 μL of 1.044 mg/mL IgG in 1x PBS. Initial concentration of IgG was determined by NanoDrop 2000 using an E1% of 13.7. As in Example 2, there was an additional plunger dispense of 50 μL with a hold time of twenty seconds after the sample was pushed into the resin bed by the insertion of the mandrel in the SizeX₁₅₀ tip. Material was eluted from the SizeX₁₅₀ column as described in Example 2. The volume of the eluted material was measured by a manual Eppendorf 200 μL pipette. The concentration of the eluted material was measured at 280 nm on a NanoDrop2000. The average eluted material was 148±1.29 μL with a concentration of 0.840±0.034 mg/mL resulting in an average yield of 79.1±2.69%. These results correspond to a 24% dilution of the initial analyte solution.

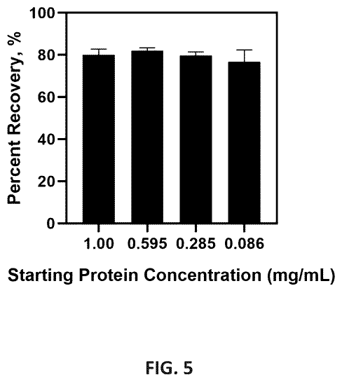

In a related example, it was demonstrated that the initial concentration of a large protein sample analyte had little bearing on the yield or dilution factor. In this example, 150 μL of IgG at four initial concentrations (1.00, 0.595, 0.285, and 0.086 mg/mL) was used as the sample of interest. The buffer exchange protocol followed as was described earlier in Example 6. Yields, as shown in FIG. 5, (n=4) were 80.0±2.44%, 82.1±1.07%, 79.6±1.70%, and 76.6±5.05% for 1.00, 0.595, 0.285, and 0.086 mg/mL, respectively. Eluted volumes were 143±2.06, 147±2.06, 146±2.38, and 145±1.50 μL for the same concentrations, respectively. The final concentrations (and dilutions) for each of the four concentrations were 0.837±0.019 mg/mL (19%), 0.499±0.012 mg/ml (19%), 0.234±0.007 mg/mL (22%), and 0.063±0.005 mg/mL (38%), respectively.

### Example 7: Recovery of Biological Samples of Various Sizes Using SizeX₁₀₀ and SizeX₁₅₀ Tips on a Dynamic Devices Automated Liquid Handling System

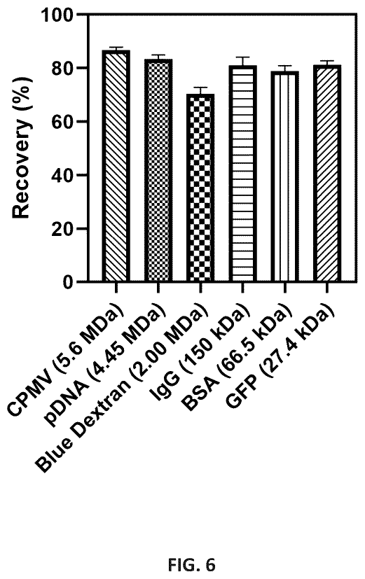

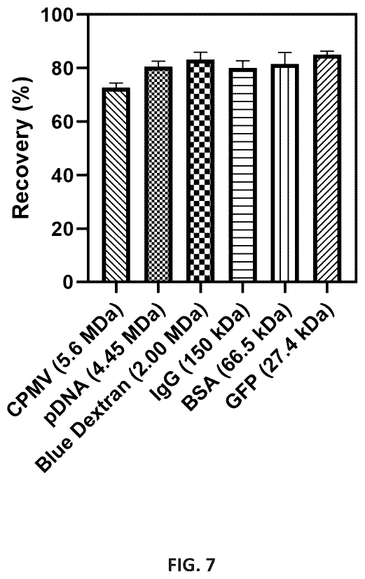

In this example, biological samples ranging in size from 66.5 kDa to 5.60 MDa were used to demonstrate biological molecules of different sizes and compositions could be buffer exchanged while maintaining a similar concentration at high yield using both SizeX₁₀₀ and SizeX₁₅₀ tips on a Dynamic Devices Lynx LM1200. Methodology for measuring the recovery of molecules was like that described in Example 1 with a few exceptions. In this example, the materials used as a sample load were 0.95 mg/mL bovine serum albumin (66.5 kDa), 1.0 mg/mL Blue Dextran (2.00 MDa), 79 ng/μL plasmid DNA (4.45 MDa), and 1.0 mg/mL Cowpea Mosaic Virus (CPMV) (5.6 MDa). Initial concentrations of biomolecules were determined by NanoDrop 2000. SizeX₁₀₀ and SizeX₁₅₀ desalting conditions as described in Table 1 were used with appropriate samples volumes for each new biomolecule. Average yields for SizeX₁₀₀ samples (11-8) were 78.9±2.1%, 70.5±2.4%, 83.4±1.6%, and 86.8±1.1% for BSA, Blue Dextran, plasmid DNA, and CPMV, respectively. Average yields for SizeX₁₅₀ samples (n=6) were 81.6±3.9%, 83.2±2.5%, 80.6±1.8%, and 72.8±1.6% for BSA, Blue Dextran, plasmid DNA, and CPMV, respectively. Data was combined with data from Examples 1-4 to generate FIGS. 6 and 7.

### Example 8: Small Molecule Removal Efficiency Using SizeX₁₀₀ Tips on a Tecan Fluent Automated Liquid Handling System

In this example, a UV-vis spectrometer was used to measure the absorbance of a solution containing a small molecule (dye) prior to and after being passed through a SizeX₁₀₀ tip using the flexible channel arm (FCA) on a Tecan Fluent 1080.

The base of the SizeX₁₀₀ tips were constructed by placing a porous 0.072″ polyethylene frit at 1.5 mm above the apical orifice of a 1000 μL wide bore tip (VWR Cat #49133-0000). 262.5 mg (wet mass) of an inert resin with a 1,000-6,000 MW fractionation range (Bio-Gel P-6DG) was added to the top of the frit and allowed to settle. Tips were capped on both ends and stored at 4° C. until they were ready for use.

The Fluent system uses Tecan's proprietary software and has several notable differences than the Dynamic Devices software. As described in the background, the “Prepare Axis Move” command can be utilized to pull the plunger up for individual channels Unlike traditional pipetting commands, this value is not strictly defined as a volume. Instead, the movement of the piston is controlled as a “position”) (mm/°. A position value of 0 mm designates a fully dispensed plunger. Similarly, the movement rate of the plunger is given as a “factor” or % of the maximum speed (105 mm/s).

As in Example 1, tips could be prepared for use in the method in two manners. Prior to use on the Tecan system, tips could be centrifuged at 200×g for 1 minute. Alternatively, tips could be placed directly in a tip a rack. The plungers on the FCA could be pulled up to a height corresponding to a position of 50 mm and the SizeX₁₀₀ tips could then be picked up by the channels and the plungers fully dispensed (0 mm) over a waste trough. An additional hold time of 3 seconds allowed residual liquid to be removed. The resulting action of both methods was a packed bed and the removal of storage solution.

The general workflow used for buffer exchange is to equilibrate the column in the buffer the user wants the final product to be in, load the sample of interest, and elute the material off with a set amount of buffer. The goal is to use the length of the column and the diffusion of smaller molecules into the pores of the resin to separate the analyte of interest from molecules of different sizes present in the original sample.

In this example, a small molecule detectable by absorbance, bromophenol blue (BPB), was used to determine what percent of a small molecule could be removed from a starting sample. The absorbance of BPB, a blue dye, was measured at 591 nm on a BioTek Synergy HTX plate reader. A standard curve of BPB (1.17 μM-150 μM) was constructed using the BioTek Synergy HTX plate reader. A solution of 150 μM BPB in 1x PBS (100 μL) was loaded onto the top of a SizeX₁₀₀ column bed that had been equilibrated with 900 μL of 1x PBS. In this example, the pistons were moved 10 mm prior to SizeX₁₀₀ tip pick up by the channels. As tips matched the dimensions of standard wide-bore Tecan tips, tip pick up was treated in a traditional manner. Tips were picked up and placed over a microplate. As the pipetting head was inserted into the SizeX₁₀₀ tips and the piston position was restored to 0 mm at a factor of 10%, the BPB sample was pushed into the resin bed and liquid was displaced out of the apical ends of the SizeX₁₀₀ tips. Tips were allowed to drain for an additional 12 seconds. Tips were then placed back into the tip rack. 100 μL of chaser solution (1x PBS) was added on top of the resin bed. The plunger on the multi-channel pipetting head was extended to a position of 15 mm prior to tip pick up. Tips were then picked up and the positioned over a collection plate. The plungers were dispensed to 0 mm at a factor of 5% and tips were allowed to drain for 15 additional seconds. The eluted material was then measured at 591 nm. The concentration of BPB in the eluted material was calculated by translating the measured absorbance value at 591 nm to concentration via a standard curve. The measured absorbance of remaining BPB in the elution was 0.006±0.003 AU (n=8) yielding a concentration for the samples below the limit of quantification (1.17 μM, Abs_{λ=591 nm}=0.021 AU). This demonstrates at least a 128-fold dilution factor of BPB, equating to over >99% small molecule removal.

### Example 9: Small Molecule Removal Efficiency Using SizeX₁₅₀ Tips on a Tecan Fluent Automated Liquid Handling System

In this example, a UV-vis spectrometer was used to measure the absorbance of a solution containing a small molecule (dye) prior to and after being passed through a SizeX₁₀₀ tip using the flexible channel arm (FCA) on a Tecan Fluent 1080.

The base of the SizeX₁₅₀ tips were constructed by placing a porous 0.072″ polyethylene frit at 1.5 mm above the apical orifice of a 1000 μL wide bore tip (VWR Cat #49133-0000). 350 mg (wet mass) of an inert resin with a 1,000-6,000 MW fractionation range (Bio-Gel P-6DG) was added to the top of the frit and allowed to settle. Tips were capped on both ends and stored at 4° C. until they were ready for use.

As in Example 2, tips could be prepared for use in the method in two manners. Prior to use on the Tecan system, tips could be centrifuged at 200×g for 1 minute. Alternatively, tips could be placed directly in a tip a rack. The plungers on the FCA could be pulled up to a height corresponding to a position of 50 mm and the SizeX₁₅₀ tips could then be picked up by the channels and the plungers fully dispensed (0 mm) over a waste trough. An additional hold time of 3 seconds allowed residual liquid to be removed. The resulting action of both methods was a packed bed and the removal of storage solution.

In this example, a small molecule detectable by absorbance, bromophenol blue (BPB), was used to determine what percent of a small molecule could be removed from a starting sample. The absorbance of BPB, a blue dye, was measured at 591 nm on a BioTek Synergy HTX plate reader. A standard curve of BPB (1.17 μM-150 μM) was constructed using the BioTek Synergy HTX plate reader. A solution of 150 μM BPB in 1x PBS (150 μL) was loaded onto the top of a SizeX₁₅₀ column bed that had been equilibrated with 900 μL of 1x PBS. In this example, the pistons were moved 25 mm prior to SizeX₁₅₀ tip pick up by the channels. As tips matched the dimensions of standard wide-bore Tecan tips, tip pick up was treated in a traditional manner. Tips were picked up and placed over a microplate. As the pipetting head was inserted into the SizeX₁₅₀ tips and the piston position was restored to 0 mm at a factor of 10%, the BPB sample was pushed into the resin bed and liquid was displaced out of the apical ends of the SizeX₁₀₀ tips. Tips were allowed to drain for an additional 12 seconds. Tips were then placed back into the tip rack. 150 μL of chaser solution (1x PBS) was added on top of the resin bed. The plunger on the multi-channel pipetting head was extended to a position of 27 mm prior to tip pick up. Tips were then picked up and the positioned over a collection plate. The plungers were dispensed to 0 mm at a factor of 5% and tips were allowed to drain for 15 additional seconds. The eluted material was then measured at 591 nm. The concentration of BPB in the eluted material was calculated by translating the measured absorbance value at 591 nm to concentration via a standard curve. The measured absorbance of remaining BPB in the elution was 0.014±0.006 AU (n=8) yielding a concentration for the samples below the limit of quantification (1.17 μM, Abs_{λ=591 nm}=0.036 AU). This demonstrates at least a 128-fold dilution factor of BPB, equating to over >99% small molecule removal.

### Example 10: Recovery of Protein Samples Using SizeX₁₀₀ Tips on a Tecan Fluent Automated Liquid Handling System

An important characteristic of a buffer exchange device is to recover a high percentage of the analyte of interest without excessive dilution. In this example, a recombinant protein, his₆-tagged green fluorescent protein (GFP) (MW: 27,402 Da), was used to demonstrate a small protein could be buffer exchanged while maintaining a similar concentration at high yield using SizeX₁₀₀ tips on a Tecan Fluent 1080. Methodology for measuring the recovery of GFP was similar to that described in Example 8 with a few exceptions. In this example, the material used as a sample load was 100 μL of 1.00 mg/mL GFP in 1x PBS. Initial concentration of GFP was determined by NanoDrop 2000 using a calculated extinction coefficient of 25102 M⁻¹ cm⁻¹ according to the methodology described by Gill and von Hippel (Analytical Biochemistry, 1989, 182 (2), 319-326). A standard curve for GFP was constructed using both fluorescence (Ex_{λ}=485 nm and Em_{λ}=530 nm) and absorbance (λ=505 nm). As in Example 7, there was a 10 mm (position) plunger movement after the sample was pushed into the resin bed by the insertion of the mandrel in the SizeX₁₀₀ tip Material was eluted from the SizeX₁₀₀ column as described in Example 7. The volume of the eluted material was measured by a manual Eppendorf 200 μL pipette. The concentration of the eluted material was measured by absorbance at 505 nm on a BioTek Synergy HTX plate reader and by fluorescence (Ex_{λ}=485 nm and Em_{λ}=530 nm) on a Tecan Infinite® 200 Pro. The average volume of (n=4) eluted material was 100±0.82 μL. The average concentration when measured by absorbance was 0.842±0.046 mg/mL and the average concentration when measured by fluorescence was 0.860±0.093 resulting in average yields of 84.2±4.60% and 86.0±9 30%, respectively. These results correspond to a 16% dilution of the initial analyte solution.

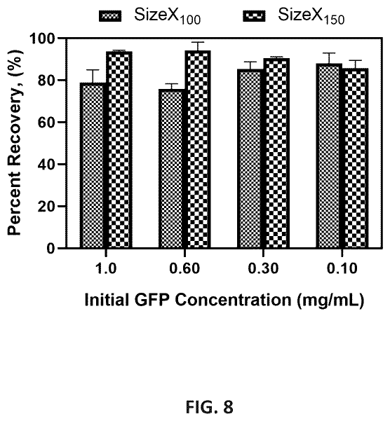

In a related example, it was demonstrated that the initial concentration of the sample analyte had little bearing on the yield or dilution factor. In this example, 100 μL of GFP at four initial concentrations (1.0, 0.60, 0.30, and 0.10 mg/mL) was used as the sample of interest. The buffer exchange protocol followed as was described earlier in Example 9. Yields, as shown in FIG. 8, (n=2) were 79.0%, 75.9%, 85.4%, and 88.0% for 1.0, 0.60, 0.30, and 0.10 mg/mL, respectively. The final dilutions for each of the four concentrations were 27%, 32%, 17%, and 14%, respectively.

### Example 11: Recovery of Protein Samples Using SizeX₁₅₀ Tips on a Tecan Fluent Automated Liquid Handling System

An important characteristic of a buffer exchange device is to recover a high percentage of the analyte of interest without excessive dilution. In this example, a recombinant protein, his₆-tagged green fluorescent protein (GFP) (MW: 27,402 Da), was used to demonstrate a small protein could be buffer exchanged while maintaining a similar concentration at high yield using SizeX₁₅₀ tips on a Tecan Fluent 1080. Methodology for measuring the recovery of GFP was similar to that described in Example 9 with a few exceptions. In this example, the material used as a sample load was 150 μL of 1.00 mg/mL GFP in 1x PBS. Initial concentration of GFP was determined by NanoDrop 2000 using a calculated extinction coefficient of 25102 M⁻¹ cm⁻¹ according to the methodology described by Gill and von Hippel (Analytical Biochemistry, 1989, 182 (2), 319-326). A standard curve for GFP was constructed using absorbance (λ=505 nm). As in Example 8, there was a 25 mm (position) plunger movement after the sample was pushed into the resin bed by the insertion of the mandrel in the SizeX₁₅₀ tip. Material was eluted from the SizeX₁₅₀ column as described in Example 8. The volume of the eluted material was measured by a manual Eppendorf 200 μL pipette. The concentration of the eluted material was measured by absorbance at 505 nm on a BioTek Synergy HTX plate reader. The average volume of (n=4) eluted material was 149±1.16 μL. The average concentration when measured by absorbance was 0.968±0.057 mg/mL resulting in an average yield of 96.2±6.20%. These results correspond to only a 3.2% dilution of the initial analyte solution.

In a related example, it was demonstrated that the initial concentration of the sample analyte had little bearing on the yield or dilution factor. In this example, 150 μL of GFP at four initial concentrations (1.0, 0.60, 0.30, and 0.10 mg/mL) was used as the sample of interest. The buffer exchange protocol followed as was described earlier in Example 10. Yields, as shown in FIG. 8, (n=2) were 93.8%, 94.2%, 90.5%, and 85.7% for 1.0, 0.60, 0.30, and 0.10 mg/mL, respectively. The final dilutions for each of the four concentrations were 6.6%, 6.1%, 11%, and 17%, respectively.

### Example 12: Small Molecule Removal Efficiency Using SizeX₁₀₀₀ Tips on a Dynamic Devices Automated Liquid Handling System

In this example, a UV-vis spectrometer was used to measure the absorbance of a solution containing a small molecule (dye) prior to and after being passed through a SizeX₁₀₀₀ tip using the multi-channel head on a Dynamic Devices Lynx LM1200.

The base of the SizeX₁₀₀₀ tips were constructed by placing a porous “bullet-shaped” polyethylene frit (OD: 4.25 mm) whose top is 13 mm above the apical orifice of a 5000 μL wide bore tip (Dynamic Devices, Arizona). A polypropylene ring (OD: 5.5 mm) is then placed directly on top of the frit to lock it into place. 2600 mg (wet mass) of an inert resin with a 1,000-6,000 MW fractionation range (Bio-Gel P-6DG) was added to the top of the frit and ring combo and allowed to settle. Tips were capped on both ends and stored at 4° C. until they were ready for use.

Tips were prepared for use in the method in either of two manners. Prior to use on the Lynx system, tips were centrifuged at 200×g for 1 minute. Alternatively, tips were placed directly in a tip rack. The plungers on the multi-channel heads were pulled up to a height corresponding to 4000 μL and the SizeX₁₀₀₀ tips were then picked up by the multi-channel head and the plungers fully dispensed. The resulting action of both methods was a packed bed and the removal of storage solution.

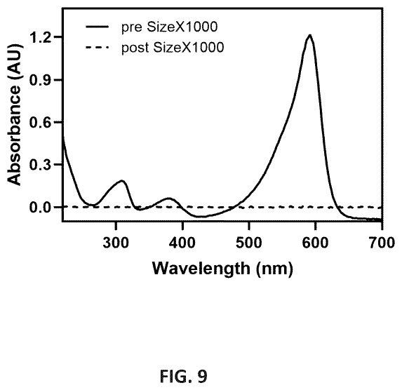

In this example, a small molecule detectable by absorbance, BPB, was used to determine what percent of a small molecule could be removed from a starting sample. The absorbance of a 0.125 mg/mL solution of BPB, a blue dye, was measured at 591 nm on a NanoDrop 2000. In this example, there was no additional piston displacement after tips were picked up. Equilibration, sample, and chaser steps were conducted using the values provided in Table 3. Tips were picked up at a height of 4.3 relative to the deck position, resulting in the insertion of the mandrel of the pipetting head at least 9.5 mm into the tops of the SizeX₁₀₀₀ tips. As the pipetting head was inserted into the SizeX₁₀₀₀ tips, the BPB sample was pushed into the resin bed and liquid was displaced out of the apical ends of the SizeX₁₀₀₀ tips. Tips were then placed back into the tip rack 1050 μL of chaser solution (phosphate buffered saline) was added on top of the resin bed. The plunger on the multi-channel pipetting head was extended to a volume of 1050 μL prior to tip pick up. Tips were then picked up and positioned over a collection plate. The plungers were dispensed and tips were allowed to drain for 60 additional seconds. The eluted material was then measured at 591 nm. Absorbance of the eluted material was 0.25% that of the starting sample, equating to greater than 99% removal of BPB from solution. The starting and resulting chromatograms are shown in FIG. 9.

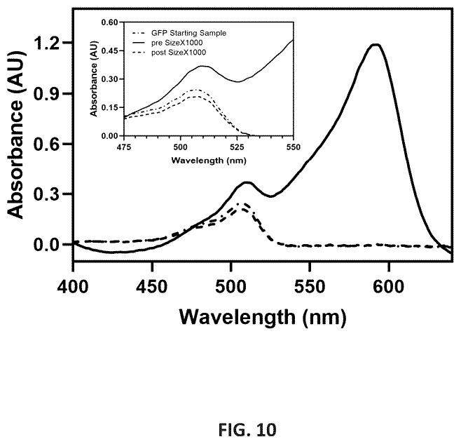

A follow-up experiment using a solution containing both 0.125 mg/mL BPB and 1 mg/mL was conducted using the same parameters. Four samples of GFP and BPB were loaded onto equilibrated SizeX₁₀₀₀ tips and eluted as described above. The initial sample, a 1 mg/mL sample of GFP, and the resulting eluate were measured on a NanoDrop 2000. The resulting chromatograms are shown in FIG. 10. Average recoveries of GFP were 84.9±2.1% (n=4). Levels of BPB remaining in solution were below the limit of detection and thus >99%.

### Example 13: Recovery of Protein Samples Using SizeX₁₀₀₀ Tips on a Dynamic Devices Automated Liquid Handling System

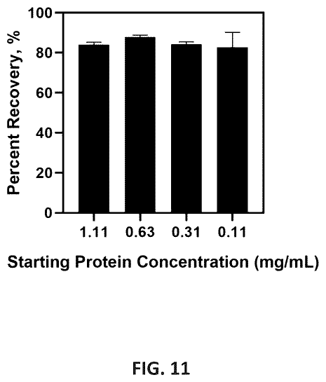

An important characteristic of a buffer exchange device is to recover a high percentage of the analyte of interest without excessive dilution. In this example, a recombinant protein, his₆-tagged green fluorescent protein (GFP) (MW: 27,402 Da), was used to demonstrate a small protein could be buffer exchanged while maintaining a similar concentration at high yield using SizeX₁₀₀₀ tips on a Dynamic Devices Lynx LM1200. Methodology for measuring the recovery of GFP was similar to that described in Example 12 with a few exceptions. In this example, the material used as a sample load was 1000 μL of a GFP solution ranging in concentration from 0.11 mg/mL to 1.11 mg/mL in 1x PBS. Initial concentration of GFP was determined by NanoDrop 2000 using a calculated extinction coefficient of 25102 M⁻¹ cm⁻¹ according to the methodology described by Gill and von Hippel (Analytical Biochemistry, 1989, 182 (2), 319-326). In this example, there was a 350 μL plunger movement after the sample was pushed into the resin bed by the insertion of the mandrel in the SizeX₁₀₀₀ tip. Material was eluted from the SizeX₁₀₀₀ column as described in Example 12. The volume of the eluted material was measured by a manual Eppendorf 200 μL pipette. The concentration of the eluted material was measured by absorbance at 280 nm on a NanoDrop 2000. The average volume across all elutes (N=16) was 1034±14.6 μL. The average elution volume did not vary significantly by concentration (n=4), with volumes of 1028±14.1 μL, 1025±18.3 μL, 1043±10.8 μL, and 1041±8.7 μL for 1.11 mg/mL, 0.63 mg/mL, 0.31 mg/mL, and 0.11 mg/mL, respectively. Yields, as shown in FIG. 11, (n=4) were 84.0±1.3%, 87.9±1.0%, 84.2±1.3%, and 82.8±7.5% for 1.11, 0.63, 0.32, and 0.11 mg/mL, respectively. The final dilutions for each of the four concentrations were 18%, 14%, 19%, and 24%, respectively.

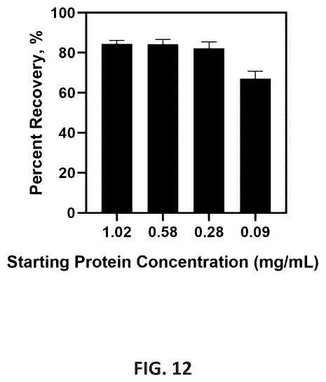

Similarly, this experiment was performed with a larger protein, polyclonal IgG (150 kDa). The concentration of the starting material and eluted material was measured by absorbance at 280 nm on a NanoDrop 2000 using an E1% of 13.7. The average volume across all elutes (N=16) was 1033±14.1 μL. The average elution volume did not vary significantly by concentration (n=4), with volumes of 1031±8.5 μL, 1038±19.4 μL, 1034±14.2 μL, and 1030±17.1 μL for 1.01 mg/mL, 0.58 mg/mL, 0.28 mg/mL, and 0.09 mg/mL, respectively. Yields, as shown in FIG. 12, (n=4) were 84.4±1.8%, 84.3±2.42%, 82.3±3.3%, and 67.1±3.4% for 1.01 mg/mL, 0.58 mg/mL, 0.28 mg/mL, and 0.09 mg/mL, respectively. The final dilutions for each of the four concentrations were 18%, 19%, 20%, and 35%, respectively.

1. A method of buffer exchange for a sample, the method comprising:
providing a pressure-fitted automated liquid handler (PF-ALH) and a plurality of buffer exchange pipette tips situated in proximity to the PF-ALH,
wherein the buffer exchange pipette tips comprise a housing comprising (i) a proximal end comprising a lower opening; (ii) a distal end designed to fit the PF-ALH; and (iii) an inert resin for size exclusion chromatography (SEC) within the housing;
using the PF-ALH to dispense the sample on top of the inert resin inside the buffer exchange pipette tip;
using the PF-ALH to apply means for creating a pressure differential; and
using the PF-ALH to engage the distal end of the buffer exchange pipette tip to facilitate movement of the sample through the inert resin;
to thereby buffer exchange the sample.

2. The method of claim 1, which further comprises, prior to dispensing the sample on top of the inert resin:
using the PF-ALH to dispense an equilibrium buffer on top of the inert resin in the buffer exchange pipette tip;
using the PF-ALH to apply means for creating a pressure differential; and
using the PF-ALH to engage the distal end of the buffer exchange pipette tip to facilitate movement of the equilibrium buffer through the inert resin.

3. The method of claim 1, which further comprises, after movement of the sample through the inert resin:
using the PF-ALH to dispense an elution buffer on top of the inert resin in the buffer exchange pipette tip;
using the PL-ALH to apply means for creating a pressure differential; and
using the PF-ALH to engage the distal end of the buffer exchange pipette tip to facilitate movement of the elution buffer through the inert resin;
to thereby elute the sample from the inert resin into the elution buffer.

4. The method of claim 1, wherein the PF-ALH comprises a plunger and the means for creating a pressure differential comprises moving the plunger prior to engaging the distal end of the buffer exchange pipette tip.

5. The method of claim 1, wherein the inert resin is comprised of a base material selected from the group consisting of agarose, cellulose, dextran, polystyrene, polyacrylamide, polyacrylate, Sephadex, or Sepharose.

6. The method of claim 1, wherein the inert resin is Sephadex, Bio-gel P or Sepharose.

7. The method of claim 1, wherein the housing comprises a frit inside the housing positioned above the lower opening and the inert resin is positioned within the housing above the frit.

8. The method of claim 1, wherein the buffer exchange pipette tip is an IMCStips® SizeX₁₀₀ tip.

9. The method of claim 1, wherein the buffer exchange pipette tip is an IMCStips® SizeX₁₅₀ tip.

10. The method of claim 1, wherein the buffer exchange pipette tip is an IMCStips® SizeX₇₅₀ tip.

11. The method of claim 1, wherein the buffer exchange pipette tip is an IMCStips® SizeX₁₀₀₀ tip.

12. The method of claim 1, wherein the sample comprises a protein.

13. The method of claim 12, wherein the protein >20 kDa.

14. The method of claim 12, wherein the protein is >100 kDa.

15. The method of claim 1, wherein the sample comprises a nucleic acid.

16. The method of claim 15, wherein the nucleic acid comprises plasmid DNA.

17. The method of claim 1, wherein the sample comprises viral particles or lipid nanoparticles.

18. The method of claim 1, wherein the PF-ALH is a Dynamic Devices® PF-ALH.

19. The method of claim 1, wherein the PF-ALH is a Tecan® PF-ALH.

20. A kit for buffer exchange comprising a plurality of buffer exchange pipette tips designed for use with a pressure-fitted liquid handler (PF-ALH) packaged with instructions for use of the buffer exchange pipette tips with the PF-ALH according to the method of claim 1.
